(19) United States
(12) Patent Application Publication
(10) Pub. No.: US 2006/0068501 A1
(43) Pub. Date: Mar. 30, 2006

(54) Apparatus and methods for steroid hormone testing

(75) Inventors: Rena Li, Scottsdale, AZ (US); LiBang Yang, Portland, OR (US); Yong Shen, Scottsdale, AZ (US)

Correspondence Address: CROWELL & MORING LLP;INTELLECTUAL PROPERTY GROUP, P.O. BOX 14300, WASHINGTON, DC, 20044-4300, US

(73) Assignee: NJ INTERNATIONAL LLC, Scottsdale, AZ (US)

(21) Appl. No.: 10/952,750

(22) Filed: Sep. 30, 2004

(51) Int. Cl. G01N 33/558

(52) U.S. Cl. 436/514

(57) ABSTRACT

An immuno-chromatographic detection device for detecting an analyte in sample, such as estrogen in a urine or saliva sample, the device comprising (a) a binding membrane having immobilized thereon (i) an test antibody against said analyte in at least one detection region, and (ii) a control antibody against a control antigen known to be present in the sample in a control region, (b) a sample membrane located at a first end of the binding membrane for receiving the sample, wherein the sample membrane is in chromatographic connection with the binding membrane, and (c) a label membrane containing (iii) a labeled antigen that is capable of binding to the test antibody and upon binding with the test antibody exhibits an observable change at the at least one detection region, and (iv) a labeled control antigen that is capable of binding to the control antibody and upon binding with the control antibody exhibits an observable change at the control region, wherein the sample membrane is separated from the label membrane by a waterproof membrane which is removable to allow the sample membrane and label membrane to be connected chromatographically. Also provided are kits comprising the device, method for detecting the analyte, and methods for manufacturing the device and kit.

### FIELD OF THE INVENTION

This invention relates to apparatus and methods for determining the concentration of a steroid hormone in a human subject.

### BACKGROUND OF THE INVENTION

Estrogens are small-molecule steroids synthesized in a number of human cells and tissues, such as ovarian granulose cells, placental syncytiotrophoblast, adipose tissue and the brain. Estrogens include typically estradiol, estriol, and estrone. Estradiol, the principal hormone of the ovary, is important for female sexual differentiation during gestation, sexual development at the onset of puberty, and regulation of the menstrual cycle. Estradiol plays roles in the menstrual cycle and involved in fertilization by both the stimulation and inhibition of the release of the gonadotropins, exerting both a positive and a negative feedback. Early in the follicular phase, ovarian secretion of estradiol from the thecal and granulosa cells is modest. During the follicular phase, estradiol stimulates endometrial growth (repairing the endometrium after menses). Toward mid-cycle, luteinizing hormone (LH) production increases and results in the release of the ovum by the rupture of the developed follicle. After ovulation, estradiol secretion declines slightly. During the luteal phase, estradiol along with progesterone are secreted by the corpus luteum, stimulating further endometrial growth. If the ovum is not fertilized, there is a further drop in estradiol and progesterone. This drop in estradiol and progesterone initiates menses.

Estradiol levels are lowest at menses during the early follicular phase (25-75 pg/mL). The levels rise in the late follicular phase to a peak of 200-600 pg/mL just before the LH surge initiates ovulation. As LH peaks, estradiol begins to decrease before rising again during the luteal phase (100-300 pg/mL). If conception does not take place, estradiol falls further to its lowest levels, thus initiating menses. If conception occurs, estradiol levels continue to rise, reaching levels of 1-5 ng/mL during the first trimester, 5-15 ng/mL during the second trimester, and 10-40 ng/mL during the third trimester. In menopausal women, estradiol levels remain low (<50 pg/ml).

Estriol levels are usually measured for pregnant women to determine the likelihood of the fetus having Down syndrome or other birth defects. The test for estriol and other estrogens is combined with tests for alpha-fetoprotein (AFP) and human chorionic gonadotropin (hCG). The three tests done together are called a triple test. The amount of estriol in the blood increases during pregnancy. It is produced in large amounts by the placenta, the tissue that links the fetus to the mother. Estriol can be detected as early as the 9th week of pregnancy, and its levels increase until delivery. Estriol can also be measured in urine.

Estrone may be measured in women who have gone through menopause to determine their estrogen levels. It may also be measured in men or women (in a urine sample) as part of a total estrogen value when a tumor of the ovaries, testicles, or adrenal glands may be present.

Estrogen produced by the ovaries helps prevent bone loss and works together with calcium and other hormones and minerals to help build bones. Human body constantly builds and remodels bone through a process called resorption and deposition. Up until around age 20, the body makes more new bone than it breaks down. But once estrogen levels start to decline, this process also slows down. In postmenopausal women, body breaks down more bone than it rebuilds. In the years immediately after menopause, women risk losing as much as 20 percent of their bone mass. Although bone loss eventually levels out more than five years post-menopause, keeping bone structures strong and healthy to prevent osteoporosis becomes more of a challenge. Osteoporosis occurs when bones become too weak and brittle to support normal activities.

Estrogens also play roles in prevention of cardiovascular disease by helping protect against plaque buildup in arteries. Estrogen does this by helping to raise HDL cholesterol (good cholesterol), which helps remove LDL-cholesterol (the type that contributes to the accumulation of fat deposits called plaque along artery walls). Postmenopausal women have a higher risk for developing coronary artery disease (CAD)—a condition in which the veins and arteries that take blood to the heart become narrowed or blocked by plaque—increases steadily. Heart attack and stroke are caused by atherosclerotic disease, in most cases. Twenty five percent of all American women have blood cholesterol levels high enough to pose a serious risk for coronary heart disease, according to the American Heart Association.

The long-term health consequences of estrogen decline after menopause includes bone loss, increased risk of cardiovascular disease, and cognitive impairment. Early diagnosis of menopause and estrogen replacement therapy (ERT, or hormone replacement therapy, HRT) may prevent those diseases.

ERT has been commonly used in US for more than 50 years. It is believed that ERT helps postmenopausal women to prevent bone lose, memory decline, and heart diseases. Studies indicated that less osteoporosis and heart diseases were reported in the women with ERT. More interestingly, these beneficial effects of estrogen were more effective in the women who started the ERT immediately after menopause than those started later.

Recently, however, it was reported that women who started ERT at a later age are under an increased risk for developing breast cancer. This implies that accurate determination of the onset of menopause, via the measurement of estradiol level, and timely start of ERT would be beneficial to women's health. Furthermore, the measurement of estradiol is important for the evaluation of normal sexual development (menarche) and causes of infertility (anovulation, amenorrhea, dysmenorrhea). Therefore, there is a need for method to accurately and rapidly monitoring estrogen level in women with adequate sensitivity.

Estrogen is also present in males and in youth, small amounts of estrogen reduce the cell-stimulating effects of testosterone. But due to aging, body fat, hormonal replacement, pesticides, nutritional deficiencies, prescription medications and excessive alcohol intake many men experience high levels of estrogen which are detrimental to their health. A testosterone/estrogen imbalance directly causes many of the debilitating health problems associated with normal aging. Thus, there is also a need to monitor estrogen levels in men.

Because their high bioactivities, steroids are present in the body at very low concentrations. Therefore, accurate detection of steroids in biological samples generally require highly sensitive methods. Detecting estrogens in a biological fluid from postmenopausal women, children or men is particularly demanding on the method's sensitivity where concentrations at the low pg/ml level are encountered.

Furthermore, it is highly desirable that a sensitive method can be performed conveniently by a person without specialized training or equipment. It is still more desirable if such detection is inexpensive, rapid and reliable.

Various methods are available for measuring estradiol levels in serum. Many of these methods utilize radioactive elements as labels and suffer from several disadvantages. Several of these methods are reviewed in U.S. Pat. No. 5,342,760 which is incorporated herein by reference. U.S. Pat. No. 5,342,760 discloses and claims a useful method for determination of estradiol in fluid samples by competitive immunoassay, but these methods typically require professional training or specialized equipment, and take a long time to complete.

### SUMMARY OF THE INVENTION

The invention generally provides a device for detecting an analyte in sample, wherein the device comprises (a) a binding membrane having immobilized thereon (i) an test antibody against said analyte in at least one detection region, and (ii) a control antibody against a control antigen known to be present in the sample in a control region, (b) a sample membrane located at a first end of the binding membrane for receiving the sample, wherein the sample membrane is in chromatographic connection with the binding membrane, and (c) a label membrane containing (iii) a labeled antigen that is capable of binding to the test antibody and upon binding with the test antibody exhibits an observable change at the at least one detection region, and (iv) a labeled control antigen that is capable of binding to the control antibody and upon binding with the control antibody exhibits an observable change at the control region, wherein the sample membrane is separated from the label membrane by a waterproof membrane which is removable to allow the sample membrane and label membrane to be connected chromatographically.

In a preferred embodiment, the test device further comprises an absorption pad located at a second end of the binding membrane opposite to the first end, wherein the absorption pad provides capillary suction to allow chromatographic migration of substances from the first end to the send end. More preferably, the device further comprises a support substrate to which the absorption pad, the binding membrane, the sample membrane, the waterproof membrane, and the label membrane are attached.

In another embodiment, the binding membrane comprises at least two detection regions adequately separated from each other, each of which contains a predetermined amount of the test antibody, wherein the occurrence, or lack thereof, of an observable change in one or more of the detection region provides a quantification of the analyte in the sample. Preferably, the binding membrane comprises four detection regions.

A test device of the present invention is preferably for detecting a steroid hormone in a fluid biological sample, especially for detecting estrogen in a saliva or urine sample. A particularly preferably test device of the present invention is for the detection and quantification of estradiol.

Many widely present antigens can be used as the control antigen, which according to a preferred embodiment is an IgG protein, and the control antibody is an anti-IgG antibody, such as a rabbit-anti-human IgG antibody.

A specific embodiment of the present invention is a test device wherein the analyte is estradiol, the fluid sample is urine or saliva, the control antigen is an IgG protein and the control antibody is an anti-IgG antibody, and the four test regions are shaped as parallel straight lines about 1-3 mm thick and 2-3 mm apart, and are numbered detection lines 1, 2, 3 and 4 starting from the first end of the binding membrane, wherein detection lines 1, 2, 3, 4 contains about 0.3-0.6, 1.0-1.7, 2.6-3.9, and 4.4-5.6 μg of anti-estradiol antibody, respectively.

The present invention also provides for a method for detecting an analyte in a fluid sample using the device of claim 1, the method comprising (1) applying a suitable amount of a sample suspected of containing the analyte to the sample membrane, (2) removing the waterproof membrane to allow the sample membrane and label membrane to be connected chromatographically, (3) optionally applying a suitable mobile phase to the label membrane to allow the labeled antigen to migrate through the binding membrane, wherein the occurrence of an observable change in the control region indicates the success of the detecting, and the absence of an observable change in at least one of the detection regions indicates the absence of a detectable amount of the analyte. Preferably, according to the present inventive method, the device further comprises an absorption pad located at a second end of the binding membrane opposite to the first end, wherein the absorption pad provides capillary suction to allow chromatographic migration of substances from the first end to the send end.

Also provided is a method for monitoring estrogen level in a mammal, the method comprising (1) obtaining a urine or saliva sample from said mammal, and (2) determining the content of estradiol in said sample according to the above inventive detection.

The present invention further provides a kit for monitoring estrogen level in a mammal, which kit comprises an inventive detection device of the present invention, a suitable container, and instruction for using the device.

The present invention also provides for a method for manufacturing the detection device and detection kit.

Other objects, advantages and novel features of the present invention will become apparent from the following detailed description when considered in conjunction with the accompanying drawing herein.

### BRIEF DESCRIPTION OF THE DRAWINGS

FIG. 1 is a side view of an immuno-chromatographic analysis test strip of the invention.

FIG. 2 is a front view of an immuno-chromatographic analysis test strip of the invention.

### DETAILED DESCRIPTION OF PREFERRED EMBODIMENTS

This invention provides, for the first time, a simple, inexpensive, rapid, sensitive, and easy-to-perform detection method, and reagents, devices and kits suitable for home-test or self-test of steroid hormones, such as estradiol, in a biological sample, especially a fluid sample, using a two-step competitive immuno-chromatographic strip. The biological samples suitable for the test generally do not need to be pretreated before the test.

Because antibody-antigen binding is highly specific, antibody-antigen reaction has been used to develop semi-quantitative or quantitative assay to measure antibody or antigen. One of the most commonly used detection method is to conjugate a microparticle to antibody or antigen for a visible antigen-antibody reaction (Beesley J 1989 “Colloidal Gold. A new perspective for cytochemical marking”. Royal Microscopical Society Handbook No 17. Oxford Science Publications. Oxford University Press.).

The present invention takes advantage of the competitive immunoassay approach to achieve increased sensitivity. An analyte antigen (to be detected or quantified) in a sample competes with a labeled antigen for the same antibody which is affixed on a suitable substrate at a predetermined location(s) and amount(s). The binding between the labeled antigen and the antibody produces an observable change (e.g. a color reaction) which indicates the presence and/or amount of the analyte antigen. When the analyte antigen is absent or present in a low concentration, more antibody would be available for binding to the labeled antigen, and a stronger reaction is observed. When the analyte antigen in the sample is present at a high concentration, less antibody would be available to react with the labeled antigen and the reaction (e.g. color change) will be weak.

In general, competitive diagnostic immunoassays require a labeled immuno-reactant antigen that can compete with the analyte antigen for available antibody sites. Examples of labels include colloid gold, colored latex particle, carbon particles or fluorescent, luminescent, radioactive particles and enzymes conjugated with the immuno-reactant. Preferably for the present invention, such a label will produce a reaction that is directly observable without the assistance of expensive equipment or specialized training. In a particularly preferred embodiment, the present invention utilizes colored particles, such as colloid gold particles, silver enhanced colloid gold particles, or colored latex particles. A colloid-gold labeled antigen is preferred which, upon reaction with a suitable antibody, produces a color change that is easily observable by the naked eye.

Here, we used antigen conjugation in this invention. Successful creation of antigen conjugates depends on two factors: size and the situation of the amino acid residues that control the binding of the antigen to the label (e.g. colloid). For a rapid assay such as competition assays, as much antigen conjugates as possible should be used, are. For such assay, a colloid gold is often chosen.

For antigens with a small molecule, such as those with a molecular weight of less than 30 kDa, or without the binding residues, an efficient solution is to preconjugate the antigen to a carrier molecule, such as bovine serum albumin (BSA) or keyhole limpet hemocyanin (KLH), followed by colloid gold conjugation. It is known to those skilled in the art that one must consider the type of linker used, the length of the linker used, the molar ratio of the hapten to the BSA, and the type of carrier molecule used, in order to carry out a reproducible conjugation that exposes the working reactive epitopes of the antigen—and therefore maximizes the potential sensitivity of the protein-carrier-gold conjugate in the assay.

In a preferred embodiment, the competitive assay device of the present invention comprises a chromatographic material suitable for the above described immuno-competitive assays. For example, a suitable material is a membrane made of nitrocellulose, a glass fiber, nylon. Preferably, the pore size of the membrane is about 2˜10 μm, with a thickness of about 50˜300 μm.

According to the method of the present invention, a sample containing the analyte antigen is applied to this material, at a region that is away from the site where a suitable antibody is immobilized on the same material. The analyte is then brought into contact with the immobilized antibody, for example via chromatographic migration by action of a mobile phase, e.g. water or a suitable buffer present in the sample or applied later. Subsequently, pre-labeled antigen is brought into contact with the rest, unreacted antibody, if any is left from the reaction with the antigen analyte in the sample. Again, the binding of the pre-labeled antigen with the immobilized antibody produces an observable reaction which in turn indicates the presence or absence of the analyte antigen in the sample. The concentration or amount of the antibody and the locations where it is immobilized on the chromatographic material are carefully controlled such that the quantity of the analyte antigen can also be determined.

Chemiluminescent techniques are also suitable for labeling and detection for the present invention. In chemiluminescent assays, luminescent compounds emit light during the course of a chemical reaction. The labels used for such assays are commonly luminol derivatives or acridinium esters. The kinetics of assays using chemiluminescence are very fast, and light is emitted within seconds of substrate oxidation. In an electrochemiluminescence (ECL) technique, a ruthenium metal chelate and tripropylamine are utilized (see e.g. Yang et al., “Electrochemiluminescence: A New Diagnostic and Research Tool,” BioTechnology 12 (1994): 193-194. and Jameison et al., “Electrochemiluminescence-Based Quantitation of Classical Clinical Chemistry Analytes,” Analytical Chemistry 68 (1996): 1298-1302.) Both of these molecules become oxidized at the surface of an electrode, where they react to form an excited state of ruthenium that decays, releasing a photon at 620 nm.

Referring now to the drawings, particularly preferred embodiments of the present invention are described in more detail below.

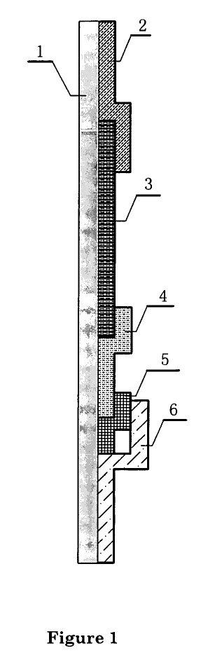

Referring to FIG. 1, the device of the present invention comprises a chromatographic material (3) suitable for immuno-competitive assays. This component is hereinafter referred to as the “binding membrane.” Suitable binding membranes include nitrocellulose membrane, as discussed above.

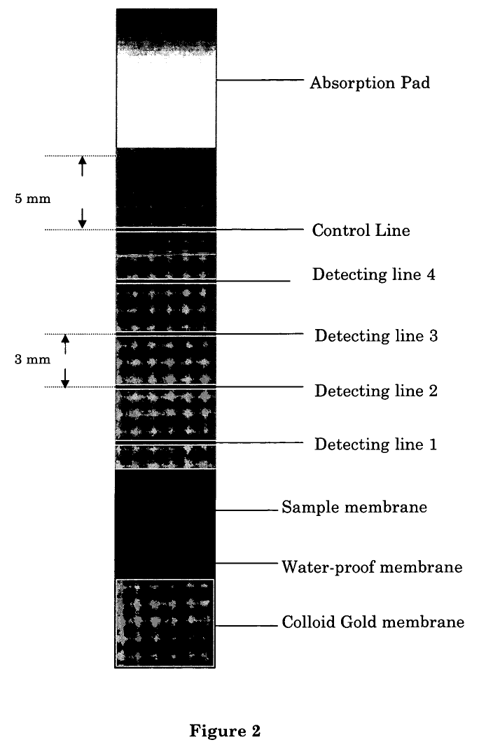

On the binding membrane 3, a suitable number of regions (marked as “Detecting Lines” in FIG. 2 are immobilized with different concentrations of one or more specific antibody that would bind with the analyte antigen to be detected. These regions are adequately and suitably separated from each other, to avoid signals from diffusing and obscuring each other during manufacturing and testing. In addition, a control antibody (“Control,” see FIG. 2), specific against an antigen known to be present in the sample, is also immobilized, preferably at a distal end from where the sample is to be applied on the binding membrane. The Control reaction ensures that the test device of the present invention functions properly and that the test has been conducted correctly. If the Control does not show a color change, for example, the test has failed, for any number of possible reasons and the test must be repeated, with fresh test devices or samples. For convenience of description, the Detecting Lines are consecutively numbered from the end the end closest to the sample (Detecting Line 1, 2, etc., see FIG. 2).

Any common antigen known to always exist in the sample can be used as the control. For example, IgG is known to exist in most body fluid samples. Accordingly, a pre-labeled (e.g. with colloid gold) IgG can be used as the control pre-labeled antigen, and anti-IgG antibody, e.g. a rabbit anti-human IgG, can be immobilized at the Control Line (see FIG. 2) as the control.

In the example shown in FIG. 1 and discussed below in more details, the binding membrane is a strip of nitrocellulose of a suitable width. The sample is to be applied at the lower end of the binding membrane, and upon chromatographic migration, will move upwards, assisted by the capillary suction exerted by an Absorbent Membrane or Absorption Pad (2) at the top end of the figure. The sample, as it moves up, will encounter the immobilized antibody. The analyte antigen, if present, will bind to the antibody present in Detecting Line 1, and if any unbound antigen is left, it will continue to bind to the antibody in Detecting Line 2, and so on.

Upon completion of the migration of the sample, a labeled antigen is brought into contact with the detecting lines, and the antigen will react with the immobilized antibody, if any is left, in the detecting lines. The above arrangement allows for the quantification of the analyte antigen present in the sample. Thus, if the analyte antigen concentration is low, it will be exhausted upon binding with the antibodies in Detecting Line 1, leaving the antibodies in the Detection 2 and any subsequent detecting lines to bind with the labeled antigen, producing the observable reaction, e.g. color change. Thus, after detection, if Detecting Line 1 shows no color change, but detection line 2 does, it indicates that the sample has an analyte antigen concentration lower than if Detection Lines 1 and 2 both showed no color change, but Detecting Line 3 does.

With quantitative calibration (e.g. with the assistance of a dose response curve), the above correlation can be used to provide a more quantitative determination of the concentration of the analyte antigen in the sample. In other words, a predetermined amount of immobilized antibody at the various detecting lines, in combination with the presence or absence of a binding reaction, can be correlated with a specific concentration range of the analyte antigen in the sample.

In the example shown in FIG. 1, there are four regions, where an anti-estradiol antibody is immobilized, each of which is about 1-3 mm in length, and about 2-3 mm apart. The coating concentrations of antibody on the nitrate cellulose membrane are 0.4, 1.2, 3.0 and 4.8 μg, respectively. These detecting lines correspond to an estradiol concentration of 50, 200, 500, 1200 pg/ml according to the standard curve, which correlates the rate of 4-methylumbelliferone formation to estradiol concentration using calibrator solutions containing known estradiol concentrations and is well known to those ordinarily skilled in the art (see e.g. John R. Crowther, The ELISA Guidebook. August 2000, Humana Press). Generally, six calibrators are used to obtain a calibration curve, though more or fewer calibrators can be used, depending on the accuracy and precision of the result desired. Preferably, the calibrators contain increasing amounts of estradiol.

In one embodiment, the immuno-chromatographic device of the present invention comprises a sample membrane (see 4, FIG. 1) separate from the binding membrane. The sample suspected of containing the analyte antigen is directed applied onto the sample membrane, and then are allowed to migrate onto the binding membrane chromatographically.

In one embodiment, the immuno-chromatographic device of the present invention further comprises a labeled antigen membrane (see 6, FIG. 1, and “gold colloid membrane” in FIG. 2) separate from the binding membrane. The labeled antigen is preferably pre-loaded onto labeled antigen membrane 6. Upon completion of the migration of the sample through the binding membrane, the labeled antigen is allowed to migrate onto the binding membrane chromatographically.

Many materials are suitable for use as both sample membrane and the labeled antigen membrane, including fiber glass or other absorbent materials.

In one embodiment, the immuno-chromatographic device of the present invention comprises a sample membrane, a labeled antigen membrane, and a water-proof membrane (see 5, FIG. 1, and “water-proof membrane” of FIG. 2), which chromatographically separates the sample membrane and the labeled antigen membrane. The water-proof membrane is removed upon completion of the migration of the sample through the binding membrane, allowing the sample membrane to be in direct contact with the pre-labeled antigen membrane. Upon application of a solvent phase (e.g. water) on the pre-labeled antigen membrane, the pre-labeled antigen migrates onto the sample membrane, and then the binding membrane, to bind with the immobilized antibody on the binding membrane.

As exemplified in FIG. 1, the device of the present invention may also comprise a suitable substrate support member that supports the other components of the device. For both the support member and the water-proof membrane, any plastic sheet with appropriate strength, flexibility and/or thickness is suitable.

It is recognized that any absorbent material is suitable for use as the absorption membrane or absorption pad 2. According to one aspect of the invention, the materials and its size (volume) for the absorption pad is chosen such that it absorbs a pre-determined amount of liquid. Thus, the user of the device of the present invention can simply apply the sample to the sample membrane, without a need to determine the amount of sample used, because the effective amount of the sample that can move through the binding membrane is controlled by the absorption capacity of the absorption pad, thereby enabling the device to quantify the concentration of the analyte in the sample.

It has been found to be difficult to obtain antibodies that have the appropriate affinity for the analyte antigen relative, especially when the antigen needs to be conjugate with a labeling component. This is especially the case when the antigen is a small steroid molecule, such as estradiol. The use of many antibodies in a competitive assay often result in an inadequate dose response, which results in inferior sensitivity, inferior precision, or both. In addition, even when antibodies that demonstrate an appropriate affinity for an analyte can be developed, many of these antibodies may demonstrate undesirable properties, such as high cross-reactivity to structurally-similar steroids. An suitable antibody is the polyclonal anti-estradiol antibody described in the Examples below. However, many commercial antibodies are also applicable to the invention.

The method and device of the present invention can be used to detect estrogen or other steroid hormone in biological samples such as urine, saliva, blood. Preferably, the sample is a urine or a saliva sample. There is no need for pre-processing of the urine or saliva sample, making the present invention particularly advantageous for in-homer self-testing.

The present invention allows the quantification of estrogen level in saliva or urine samples with a simple step. It can be used to monitor the effect of hormone treatment, predict the menopause onset, prevent risk of high estrogen-induced breast cancer, and help to choose the best effective alternative hormone therapy.

It is readily recognized that with the suitable choice of antibody, the method, device and kit of the present invention can be used to detect any analyte antigen, especially other steroid hormones, e.g. estriol, estrone, progesterone, etc.

The present invention will now be illustrated in more detail in the following examples. It is to be understood that these examples serve only to describe the specific embodiments of the present invention, but do not in any way limit the scope of the claims.

### EXAMPLES

### Example 1

### Preparation of Binding Membrane

1. Preparation of Colloid Gold Solution

Prepare the glass vessels by soaking in 3%-10% dimethyldichlorosilane chloroform solutions for about 1 minute, air dry, washing with the distilled water, and air dry again at room temperature. Mix 80-120 ml of 0.08%-0.12% chloroauric acid solution with 0.5-0.9 ml of 0.8%-0.12% sodium citrate in a preheated glassware, heat to boiling. The solution will turn from yellow to purple. Continue to boil for 10-20 minutes. After cooling, add distilled water to bring the volume to the original (80-120 milliliter).

2. Preparation of Colloid-Gold Labeled Antigen (Estradiol)

The protocol is as follows: 1. Adjust colloid gold solution prepared in Example 1 to pH 8.2-8.6 using 0.08-0.12 M potassium acetate solution; 2. Mix 300-500 μg of antigen (estradiol) with 80-120 ml colloid gold solution for 10-15 min at room temperature; 3. Add 4-10 ml of 0.8-1.3% polyethylene glycol solution; 4. Centrifuge at 10,000˜100,000 g for 20-40 min, carefully remove the supernatant; 5. Mix the pellets with 80-120 ml of 0.2˜0.5 mg/ml polyethylene glycol buffer solution; 6. Repeat step 4 and 5; and 7. Add 30-70 ml nitrine sodium, mix, and store at 4° C.

3. Preparation of Colloid-Gold Labeled Control Antigen (Rabbit IgG)

1. Adjust colloid gold solution prepared in Example 1 to pH 7.9 using a acetate salt solution; 2. Mix 350 μg of antigen (estradiol) with about 100 ml colloid gold solution for 14 min at room temperature; 3. Add 5 ml of 1% polyethylene glycol (PEG 20000) solution; 4. Centrifuge at 10,000˜100,000 g for about 25 min, carefully remove the supernatant; 5. Mix the pellets with about 100 ml of 0.5 mg/ml polyethylene glycol buffer solution; 6. Repeat step 4 and 5; and 7. Add 30-70 ml nitrine sodium, mix, and store at 4° C.

4. Preparation of membrane containing pre-labeled analyte and control antigens

Mix 50 μl each of the pre-labeled analyte and control antigens prepared in steps 2 and 3, and apply the mixture to a suitable glass fiber membrane, dry at room temperature or at 4° C.

### Example 2

### Preparation of Nitrocellulose Binding Membrane

On a strip of nitrocellulose membrane, apply an antibody against the analyte antigen (estradiol) in three detection lines. The detection lines are about 1-3 mm wide, and are about 2-3 mm apart. From the end that is closest to the sample membrane, the coating concentrations of antibody on the nitrocellulose membrane are 0.4, 1.2, 3.0 and, 4.8 μg, respectively, which correspond to an estrogen concentration of 50, 200, 500, 1200 pg/ml in a 100 μl sample. The standard curve is established using the rate of 4 methylumbelliferone formation. A total of 4 testing lines and a 5 μg control antibody are used. A fifth line, of the anti-IgG antibody, which is also about 2-3 cm from the detecting line next to it, is also applied on the nitrocellulose membrane.

The final chromatographic test device or “strip” is prepared as shown in FIG. 1. It includes 1 as supporting plate, 2 as absorbing pad, 3 as the nitrocellulose binding membrane, 4 as the sample membrane, 5 as the water-proof membrane, and 6 as the colloid gold membrane. The nitrocellulose membrane is attached toward the mid-section of the supporting plate. The absorbing pad is attached at the top end of the supporting plate, and is chromatographically connected with the nitrocellulose membrane. The colloid gold membrane is attached at the lowermost end of the supporting plate and in contact with the water-proof membrane, but chromatographically separated from the sample membrane. The sample membrane is attached on the supporting plate and chromatographically connected with the nitrocellulose membrane on one side, and in contact with the water-proof membrane, which chromatographically separates the sample membrane from the colloid gold membrane. The length of colloid gold membrane is constructed and arranged in such a way that, when the water-proof membrane is removed, the colloid gold membrane would be able to connect chromatographically the sample membrane, allowing the colloid gold-labeled antigens (both analyte and control) to migrate through the nitrocellulose membrane and bind the antibody thereon.

### Example 3

### Detection and Quantification of Estrogen Level in a Sample Using the Chromatographic Analysis Device

A sample of about 0.1-0.5 ml saliva or urine is applied (for example, using a tube provided with the kit, 1 drop is about 50 μl), without any pre-treatment or preparation, to the sample membrane, and allowed to react for 1 min. Then the water-proof membrane between the sample membrane and the colloid gold membrane is removed, following by addition of 0.1 ml of water on the colloid gold membrane. Allow two minutes for the labeled antigens to migrate through the binding membrane. Afterwards, the nitrocellulose membrane is examined visually to determine if any of the detection line has undergone any color change (i.e. turning red). If all four detection lines turn red, then the sample contains less than 50 pg/ml of estrogen. If three four detection lines turn red, then the sample contains about 50 pg/ml of estrogen. If two detection lines turn red, then the sample contains about 200 pg/ml of estrogen. If only one detection line turns red, then the sample contains about 500 pg/ml of estrogen. If none of detection lines turns red, then the sample contains more than 1200 pg/ml of estrogen. In all cases, the control detection line must always turn red, otherwise the test has failed and must be repeated with fresh reagents and/or samples.

What is claimed is:

1. A device for detecting an analyte in sample, wherein the device comprises
(a) a binding membrane having immobilized thereon (i) an test antibody against said analyte in at least one detection region, and (ii) a control antibody against a control antigen known to be present in the sample in a control region,
(b) a sample membrane located at a first end of the binding membrane for receiving the sample, wherein the sample membrane is in chromatographic connection with the binding membrane, and
(c) a label membrane containing (iii) a labeled antigen that is capable of binding to the test antibody and upon binding with the test antibody exhibits an observable change at the at least one detection region, and (iv) a labeled control antigen that is capable of binding to the control antibody and upon binding with the control antibody exhibits an observable change at the control region,
wherein the sample membrane is separated from the label membrane by a waterproof membrane which is removable to allow the sample membrane and label membrane to be connected chromatographically.

2. A test device according to claim 1, wherein the device further comprises an absorption pad located at a second end of the binding membrane opposite to the first end, wherein the absorption pad provides capillary suction to allow chromatographic migration of substances from the first end to the send end.

3. A test device according to claim 2, wherein the device further comprises a support substrate to which the absorption pad, the binding membrane, the sample membrane, the waterproof membrane, and the label membrane are attached.

4. A test device according to claim 1, wherein on the binding membrane comprises at least two detection regions adequately separated from each other, each of which contains a predetermined amount of the test antibody, wherein the occurrence, or lack thereof, of an observable change in one or more of the detection region provides a quantification of the analyte in the sample.

5. A test device according to claim 4, wherein on the binding membrane comprises four detection regions.

6. A test device according to claim 1, wherein the analyte is a steroid hormone and the sample is a fluid biological.

7. A test device according to claim 6, wherein the steroid hormone is estrogen.

8. A test device according to claim 7, wherein the steroid hormone is estradiol.

9. A test device according to claim 7, wherein the fluid sample is urine or saliva.

10. A test device according to claim 1, wherein the control antigen is an IgG protein and the control antibody is an anti-IgG antibody.

11. A test device according to claim 7, wherein the antibody is a rabbit-anti-human IgG antibody.

12. A test device according to claim 5, wherein the analyte is estradiol, wherein the fluid sample is urine or saliva, wherein the control antigen is an IgG protein and the control antibody is an anti-IgG antibody, and
wherein the four test regions are shaped as parallel straight lines about 1-3 mm thick and 2-3 mm apart, and are numbered detection lines 1, 2, 3 and 4 starting from the first end of the binding membrane, wherein detection lines 1, 2, 3, 4 contains about 0.3-0.6, 1.0-1.7, 2.6-3.9, and 4.4-5.6 μg of anti-estradiol antibody, respectively.

13. A method for detecting an analyte in a fluid sample using the device of claim 1, the method comprising
(1) applying a suitable amount of a sample suspected of containing the analyte to the sample membrane,
(2) removing the waterproof membrane to allow the sample membrane and label membrane to be connected chromatographically,
(3) optionally applying a suitable mobile phase to the label membrane to allow the labeled antigen to migrate through the binding membrane,
wherein the occurrence of an observable change in the control region indicates the success of the detecting, and the absence of an observable change in at least one of the detection regions indicates the absence of a detectable amount of the analyte.

14. A method according to claim 13, wherein the device further comprises an absorption pad located at a second end of the binding membrane opposite to the first end, wherein the absorption pad provides capillary suction to allow chromatographic migration of substances from the first end to the second end.

15. A method according to claim 14, wherein the device further comprises a support substrate to which the absorption pad, the binding membrane, the sample membrane, the waterproof membrane, and the label membrane are attached.

16. A method according to claim 13, wherein on the binding membrane comprises at least two detection regions adequately separated from each other, each of which contains a predetermined amount of the test antibody, wherein the occurrence, or lack thereof, of an observable change in one or more of the detection region provides a quantification of the analyte in the sample.

17. A method according to claim 16, wherein the binding membrane is a nitrocellulose membrane.

18. A method according to claim 16, wherein the binding membrane comprises four detection regions.

19. A method according to claim 18, wherein the analyte is estradiol, wherein the fluid sample is urine or saliva and about 0.1-0.5 ml of the sample is applied to the sample membrane, wherein the control antigen is an IgG protein and the control antibody is an anti-IgG antibody, and wherein the labeled antigen and labeled control antigen are labeled with colloid gold,
wherein the four test regions are shaped as parallel straight lines about 1-3 mm thick and 2-3 mm apart, and are numbered detection lines 1, 2, 3 and 4 starting from the first end of the binding membrane, wherein detection lines 1, 2, 3, 4 contains about 0.3-0.6, 1.0-1.7, 2.6-3.9, and 4.4-5.6 μg of anti-estradiol antibody, respectively, and the occurrence of color change in the detection lines is used to determine the concentration of estradiol in the sample.

20. A method for monitoring estrogen level in a mammal, the method comprising (1) obtaining a urine or saliva sample from said mammal, and (2) determining the content of estradiol in said sample according to the method of claim 19.

21. A kit for monitoring estrogen level in a mammal, the kit comprises a device according to claim 1, a suitable container, and instruction for using the device.
